(19) United States
(12) Patent Application Publication
(10) Pub. No.: US 2013/0071900 A1
(43) Pub. Date: Mar. 21, 2013

(54) PROCESS FOR PROCESSING A LIGNOCELLULOSIC MATERIAL

(71) Applicant: SHELL OIL COMPANY, Houston, TX (US)

(72) Inventors: Munro MACKAY, Amsterdam (NL); Evert VAN DER HEIDE, Amsterdam (NL)

(73) Assignee: SHELL OIL COMPANY, Houston, TX (US)

(21) Appl. No.: 13/624,308

(22) Filed: Sep. 21, 2012

(30) Foreign Application Priority Data
Sep. 21, 2011 (EP) 11182216.9
Jun. 20, 2012 (EP) 12172809.1

(51) Int. Cl. D21C 3/04; C12P 7/04

(52) U.S. Cl. 435/157; 162/82

(57) ABSTRACT

Calcium-containing lignocellulosic material is converted at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated lignocellulosic material and aqueous acid solution, having an overall pH in the range from equal to or more than 3.0 to equal to or less than 4.5. The process allows reduction in the formation of insoluble salts.

The present application claims the benefit of European Patent Application No. 11182216.9 filed Sep. 21, 2011; and, European Patent Application No. 12172809.1 filed Jun. 20, 2012, the entire disclosure of which is hereby incorporated by reference.

### TECHNICAL FIELD OF THE INVENTION

The invention relates to a process for processing a lignocellulosic material. The invention further especially relates to a process for processing a calcium-containing lignocellulosic material.

### BACKGROUND TO THE INVENTION

With the diminishing supply of crude mineral oil, use of renewable energy sources is becoming increasingly important for the production of fuels and chemicals. These fuels and chemicals from renewable energy sources are often referred to as biofuels, respectively biochemicals.

Biofuels and/or biochemicals derived from non-edible renewable energy sources, such as lignocellulosic material, are preferred as these do not compete with food production. These biofuels and/or biochemicals are also referred to as second generation, renewable or advanced biofuels and/or biochemicals.

Also for the production of bio-ethanol it would be preferred to produce such from a lignocellulosic material.

WO2006/128304 explains that a first step in converting lignocellulosic material to ethanol may involve handling and possibly size reduction of the material. Hereafter the lignocellulosic material can be hydrolysed into smaller molecules, such as for example mono- or poly-saccharides.

The two primary hydrolysis processes are acid hydrolysis and enzymatic hydrolysis.

In the acid hydrolysis process, a feed may be subjected to steam and a strong acid, such as sulphuric acid. When sulphuric acid is used the acid can be concentrated (25-80 wt %) or dilute (3-8 wt %), measured as the weight of acid in the weight of acidified aqueous solution that is present with the feed.

In the enzymatic hydrolysis process, a feed may be subjected to a first acid hydrolysis step and a second enzymatic hydrolysis step. The combination of steam temperature, acid concentration and treatment time in a first acid hydrolysis step are chosen to be milder such that the cellulose surface is greatly increased, but there is little conversion of cellulose to for example glucose. Subsequently the cellulose is hydrolyzed to glucose in a second enzymatic hydrolysis step using cellulase enzymes. The first acid hydrolysis step is often referred to as pretreatment and the product of the first acid hydrolysis is often referred to as pretreated feed. Prior to the addition of enzyme in the second enzymatic hydrolysis step, the pH of the pretreated feed is adjusted to a value that is suitable for the cellulase enzymes. This typically involves the addition of alkali to increase the pH to a pH in the range from about 4 to about 6. WO2006/128304 for example mentions the addition of an acid such as 0.1 to 2 wt % sulphuric acid to the pretreatment step in the enzymatic hydrolysis process.

As explained in WO2006/128304 it is desirable to have a continuous acid pretreatment process that can be operated and maintained economically.

It is noted in WO2006/128304 that one of the factors that hinders the development of such a continuous process is that equipment downstream of a pretreatment reactor is prone to the build up of deposition of insoluble salts known as “scale”. For example, the addition of sulphuric acid to the feed during pretreatment forms mixtures of sulphuric acid, bisulphate salts and sulphate salts. Analogous salts are formed with the use of other acids, e.g. sulphite salts and bisulphite salts form after addition of sulphurous acid. The subsequent addition of alkali, after exit of the acidified feed from the pretreatment reactor, to increase the pH to a value suitable for enzyme hydrolysis or sugar fermentation increases the concentration of salts. When combined with calcium that is indigenous to the feed, the result of this increase is the formation of calcium sulphate and calcium bisulphate. These insoluble salts tend to deposit as scale on the process equipment downstream. The scale deposition can plug valves and retard the flow in the process. It increases energy requirements of the system as well as wear and tear on the pumps. It also decreases heat transfer through piping. Each of these factors contributes to a reduction of economics of the process. Although it is possible to remove the scale by washing with acid, this is a costly and time consuming process.

WO2006/128304 therefore suggests a process comprising pretreating the lignocellulosic feed at elevated pressure in a pretreatment reactor at a pH between about 0.4 and about 2.0 to produce a pressurized pretreated feed and adding one or more than one soluble base to the pressurized pretreated feed after exit from the pretreatment reactor to adjust the pressurized pretreated feed to an intermediate pH of between about pH 2.5 and 3.5 to produce a pressurized partially neutralized feed; flashing the pressurized, partially neutralized feed one or more than one time at the intermediate pH to produce a flashed feed and adjusting the pH of the flashed feed with one or more than one base to produce a neutralized feed having a pH between about 4 to about 6.

Although according to WO2006/128304 this process reduces scale deposition on process equipment, it does not reduce formation of the insoluble salts.

WO2009/145617 describes a method for treating carbohydrate-containing vegetable material with an organic acid at a temperature of at least 120° C. In its example 4 and table 5, the pH of a washed material after acid hydrolyis with lactic acid is mentioned to lie in the range from 3.11 to 5.22. The glucose yields that are obtained after enzymatic treatment, however, are low.

It would be an advancement in the art to provide a process for converting a lignocellulosic material that allows one to reduce the formation of insoluble salts. It would further be an advancement in the art to provide a process wherein such a reduction in the formation of insoluble salts could be obtained without a reduction in glucose yields after enzymatic hydrolysis.

### SUMMARY OF THE INVENTION

Accordingly, in one embodiment of the present invention provides a method for processing a lignocellulosic material comprising the steps of

a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated lignocellulosic material and aqueous acid solution, having an overall pH in the range from equal to or more than 3.0 to equal to or less than 4.5; and

b) contacting at least part of the mixture produced in step a) with a base to produce a neutralized mixture containing neutralized pretreated lignocellulosic material and one or more insoluble salts.

The pH of the mixture containing pretreated lignocellulosic material and aqueous acid solution may hereafter also be referred to as overall pH, post-reaction pH or final pH.

In another embodiment, the process may conveniently comprise an additional desalting step. In this desalting step insoluble salts may be removed. For example, in the desalting step insoluble salts may be removed from the mixture as produced in step a), the neutralized mixture as produced in step b), the neutralized pretreated lignocellulosic material as produced in step b), or a product of a subsequent step. The process according to the invention advantageously reduces the formation of insoluble salts. In the process of the invention, the amount of insoluble salts formed and the amount of insoluble salts that may need to be removed is therefore greatly decreased.

In yet another embodiment of the present invention provides a process for processing a calcium-containing lignocellulosic material comprising the steps of

i) contacting the calcium-containing lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having a pH in the range from equal to or more than 3.0 to equal to or less than 4.5, containing pretreated lignocellulosic material and one or more, preferably dissolved, calcium salts; and

ii) recovering at least part of the one or more calcium salts.

The pH of the mixture containing pretreated ligno-cellulosic material and one or more, preferably dissolved, calcium salts produced in step i) may hereafter also be referred to as overall pH, post-reaction pH or final pH. This mixture may further also contain the aqueous acid solution that remains after step i).

Further, in an embodiment according to the invention advantageously allows one to reduce the formation of insoluble calcium salts in a process for converting a lignocellulosic material to one or more sugars and/or ethanol, whilst still sufficient yields of sugar and/or ethanol can be obtained.

Therefore, in yet another embodiment of the invention provides a process for the production of at least one alkanol comprising the steps of:

a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated lignocellulosic material and aqueous acid solution, having an overall pH in the range from equal to or more than 3.0 to equal to or less than 4.5;

b) optionally contacting at least a portion of the mixture produced in step a) with a base to produce a neutralized mixture containing neutralized pretreated lignocellulosic material and one or more insoluble salts;

c) hydrolyzing at least a portion of the pretreated lignocellulosic material produced in step a) and/or at least a portion of the neutralized pretreated lignocellulosic material produced in step b) to produce a hydrolysis product; and

d) fermenting at least a portion of the hydrolysis product produced in step c) to produce a fermentation broth comprising at least one alkanol.

The pH of the mixture containing pretreated lignocellulosic material and aqueous acid solution produced in step a) may hereafter also be referred to as overall pH, post-reaction pH or final pH.

In yet another embodiment of the invention provide a method for processing a lignocellulosic material comprising the steps of:

a) contacting the lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution;

b) leaching basic compounds from the lignocellulosic material to adjust the overall pH of the mixture to a pH in the range from equal to or more than 3.0 to equal to or less than 4.5, and

c) contacting at least a portion of the mixture produced in step a) with a base to produce a neutralized mixture containing neutralized pretreated lignocellulosic material and one or more insoluble salts.

In yet further embodiment of the invention provides a process for processing a lignocellulosic material comprising the steps of

a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution, having an overall pH in the range from equal to or more than 3.0 to equal to or less than 4.5; and

b) neutralizing the mixture produced in step a) to produce a neutralized mixture having a higher pH than the mixture produced in step a).

### BRIEF DESCRIPTION OF THE DRAWINGS

The invention has been illustrated by the following non-limiting figures:

FIG. 1 shows the relation between sulphuric acid concentration and pH in an aqueous solution of sulphuric acid.

### DETAILED DESCRIPTION OF THE INVENTION

This invention relates to processes for processing a lignocellulosic material, especially a calcium-containing lignocellulosic material, comprising contacting the lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having an overall pH in the range from equal to or more than 3.0 to equal to or less than 4.5.

It was found that by contacting a lignocellulosic material, especially a calcium-containing lignocellulosic material, at a temperature in the range from equal to or more than 120° C. to equal to or less than 210° C. with an aqueous acid solution containing one or more inorganic acids and having a pH in the range from equal to or more than 1.8 to equal to or less than 4.0 in such an amount that the final pH of the mixture lies in the range from equal to or more than 3.0 to equal to or less than 4.5, and more preferably from equal to or more than 3.5 to equal to or less than 4.5, the amount of calcium salts and other insoluble salts formed can be greatly decreased.

Decreasing the concentration of calcium salts in the mixture produced in step i) may lead to an increased percentage of the calcium salts that may stay in solution and/or bound to the lignocellulosic material and/or to a decreased amount of calcium salts that may deposit. Therefore the amount of calcium salts that may need to be retrieved in step ii) can be substantially reduced.

Preferences for step i) are as described for step a) below. Preferences for step ii) are as described for the desalting step as described below.

Without wishing to be bound by any kind of theory it is believed that due to the extremely mild pH and temperature conditions a pretreated lignocellulosic material can be generated wherein at least part of the calcium and preferably all calcium remains essentially bound within the pretreated lignocellulosic material. When this calcium, which is naturally occurring in the lignocellulosic material, remains essentially bound inside the pretreated lignocellulosic material it can no longer take part in the formation of any insoluble salts, and hence the formation of insoluble salts is reduced.

Contrary to what was believed, the reaction conditions in the processes according to the invention as described above are still sufficient to obtain a pretreated lignocellulosic material that can be sufficiently hydrolysed into a hydrolysis product. In a preferred embodiment the present invention therefore also conveniently provides a process comprising contacting a lignocellulosic material, especially a calcium-containing lignocellulosic material, at a temperature in the range from equal to or more than 120° C. to equal to or less than 210° C. with an aqueous acid solution containing one or more inorganic acids having a pH in the range from equal to or more than 1.8 to equal to or less than 4.0 in such an amount that the final pH of the mixture lies in the range from equal to or more than 3.0 to equal to or less than 4.5, and more preferably from equal to or more than 3.5 to equal to or less than 4.5, to obtain a pretreated lignocellulosic material that can be hydrolysed into a hydrolysis product. Preferences for such a process are as described herein.

The “calcium-containing lignocellulosic material” in step i) and/or the “lignocellulosic material” in step a) may hereafter also be referred to as “lignocellulosic material feed” or just “feed”. In addition the term “calcium-containing lignocellulosic material” may hereafter be abbreviated as “lignocellulosic material”, as it may be considered a subclass of such lignocellulosic materials.

Calcium from such calcium-containing lignocellulosic material may form calcium salts, such as for example calcium sulphate, calcium bisulphate, calcium sulphite, calcium bisulphite, calcium carbonate or calcium acetate. Calcium salts such as calcium sulphate, calcium bisulphate, calcium sulphite, calcium bisulphite, calcium acetate and calcium carbonate can be difficult to dissolve and tend to precipitate quickly. Such calcium salts may therefore deposit as scale on the process equipment downstream, potentially causing the disadvantages as listed above.

Without wishing to be bound by any kind of theory, it is believed that when using the temperature and pH conditions as indicated above for step a), at least part of such calcium may remain bounded inside the pretreated lignocellulosic material and can no longer take part in the formation of any insoluble salts. For example, when the calcium present in a lignocellulosic material remains at least partially and preferably wholly essentially bound to—for example organic acid sites within—the lignocellulosic material, such calcium will not form any insoluble salts such as for example calcium sulphate and/or calcium bisulphate.

By a lignocellulosic material is herein understood a material containing cellulose, hemicellulose and lignin. The lignocellulosic material may be obtained from a wide variety of sources, including for example plants, forestry residues, agricultural residues, herbaceous material, municipal solid wastes, waste and recycled paper, pulp and paper mill residues, sugar processing residues and/or combinations of one or more of the above.

The lignocellulosic material can comprise for example, corn stover, soybean stover, corn cobs, corn fibre, straw (including cereal straws such as wheat, barley, rye and/or oat straw), bagasse, beet pulp, miscanthus, sorghum residue, rice straw, rice hulls, oat hulls, grasses (including switch grass, cord grass, rye grass, reed canary grass or a combination thereof), bamboo, water hyacinth, wood and wood-related materials (including hardwood, hardwood chips, hardwood pulp, softwood, softwood chips, softwood pulp and/or sawdust), waste paper and/or a combination of one or more of these.

The lignocellulosic material preferably comprises cellulose in an amount equal to or more than 20 wt %, more preferably equal to or more than 30 wt % and most preferably equal to or more than 40 wt %. For example the lignocellulosic material may comprise in the range from equal to or more than 20 wt % to equal to or less than 90 wt % cellulose, suitably in the range from equal to or more than 30 wt % to equal to or less than 80 wt % cellulose, based on the total weight of the lignocellulosic material.

Alkali metals and/or alkaline earth metals, such calcium can be naturally occurring in the lignocellulosic material. They may for example be bound to organic acid sites in the lignocellulosic material.

The lignocellulosic material may therefore be a lignocellulosic material containing one or more alkali metal(s), such as for example lithium (Li), sodium (Na) and/or potassium (K), and/or one or more alkaline earth metal(s), such as for example magnesium (Mg) and/or calcium (Ca). Preferably the lignocellulosic material is a lignocellulosic material containing calcium. That is, preferably the lignocellulosic material is a calcium-containing lignocellulosic material.

Use of the process according to the invention is especially advantageous when the lignocellulosic material is a lignocellulosic material containing equal to or more than 10 ppmw (mg/kg), preferably equal to or more than 50 ppmw, more preferably equal to or more than 100 ppmw, still more preferably equal to or more than 500 ppmw and most preferably equal to or more than 1000 ppmw of one or more alkali metal(s) and/or an alkaline earth metal(s), wherein the content in ppmw is calculated based on the total weight of the lignocellulosic material on a dry basis. By a dry basis is understood that first water is removed before the weight percentage is calculated. The content in ppmw is further calculated as an elemental weight percentage. That is, if the alkali metal and/or the alkaline earth metal is for example present as a salt, only the weight of the alkali metal and/or alkaline earth metal in the salt is taken into account.

Most preferably, the lignocellulosic material is a calcium-containing lignocellulosic material containing equal to or more than 10 ppmw, preferably equal to or more than 50 ppmw, more preferably equal to or more than 100 ppmw, still more preferably equal to or more than 500 ppmw and most preferably equal to or more than 1000 ppmw of calcium, based on the total weight of lignocellulosic material on a dry basis. As explained above ppmw (mg/kg) is calculated as the total weight in milligram of calcium element per total weight in kilogram of lignocellulosic material on a dry basis.

There is no maximum for the content of the alkali metal and/or an alkaline earth metal, but in practice most lignocellulosic materials will contain equal to or less than 50,000 ppmw of an alkali metal and/or an alkaline earth metal and/or a mixture thereof. More suitably the lignocellulosic material is a calcium-containing lignocellulosic material containing equal to or less than 50,000 ppmw of calcium, still more suitably equal to or less than 20,000 ppmw of calcium.

The process according to the inventions is especially advantageous for lignocellulosic material containing a higher percentage of alkali metals and/or alkaline earth metals, especially calcium-containing lignocellulosic materials containing a higher percentage of calcium. In a preferred embodiment the lignocellulosic material is therefore a straw, a grass or a combination thereof. More preferably the lignocellulosic material is chosen from the group consisting of wheat straw, barley straw, rye straw, oat straw, wheat grass, barley grass, oat grass, switch grass, cord grass, rye grass, reed canary grass, hardwood (such as for example birch wood), softwood and combinations thereof.

The process according to the invention may comprise one or more additional step(s) of providing the lignocellulosic material, washing the lignocellulosic material and/or reducing the particle size of the lignocellulosic material. For example, prior to step a) respectively prior to step i), the lignocellulosic material can be washed and/or reduced in particle size. Reduction of the particle size may for example be advantageous when the lignocellulosic material comprises a lignocellulosic material such as wood or straw. The particle size reduction may for example include grinding, chopping, milling, shredding, compression/expansion, crushing and/or debarking. Preferably the particle size of lignocellulosic material is reduced to a particle size in the range from equal to or more than 5 micron to equal to or less than 5 cm, more preferably in the range from 2 mm to 25 mm. The washing of the lignocellulosic material may for example comprise washing of the lignocellulosic material with water. The washing may comprise washing of the lignocellulosic material in one or more water-wash cycles, and preferably may comprise two or more water-wash cycles.

Before supplying the lignocellulosic material to step a) respectively to step i), it may further be densified, dried and/or pelletized.

In step a) respectively in step i) the lignocellulosic material is preferably contacted at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated lignocellulosic material and aqueous acid solution, having an overall pH in the range from equal to or more than 3.0 to equal to or less than 4.5.

Without wishing to be bound by any kind of theory it is believed that when contacting the lignocellulosic material with the aqueous acid solution, basic compounds may leach out of the lignocellulosic material. These leached basic compounds may neutralize part of the acid in the aqueous acid solution. It is believed that the presence of such leached basic compounds may cause the pH of the mixture of pretreated lignocellulosic material and aqueous acid solution to be higher than expected purely on the basis of the amount and concentration of aqueous acid solution added. The prior art processes compensate for this effect by adding more aqueous acid solution and/or aqueous acid solutions in a higher concentration to reach the desired pH. It is believed that by not compensating for this effect in the process according to the present invention, the formation of undesired insoluble salts in a later step can be decreased.

Hence in one embodiment step a) comprises contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated lignocellulosic material and aqueous acid solution; and leaching basic compounds from the lignocellulosic material to adjust the overall pH of the mixture to a pH in the range from equal to or more than 3.0 to equal to or less than 4.5, more preferably to a pH in the range from equal to or more than 3.5 to equal to or less than 4.5.

Analogously in a preferred embodiment step i) comprises contacting a calcium-containing lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture, containing pretreated calcium-containing lignocellulosic material, aqueous acid solution and one or more, preferably dissolved, calcium salts; and leaching basic compounds from the lignocellulosic material to adjust the overall pH of the mixture to a pH in the range from equal to or more than 3.0 to equal to or less than 4.5, more preferably to a pH in the range from equal to or more than 3.5 to equal to or less than 4.5.

Preferably such a step a), respectively such step i) is carried out in the absence of an external base.

The lignocellulosic material may therefore be contacted in step a) respectively in step i) at a temperature in the range from equal to or more than 120° C. to equal to or less than 210° C. with an aqueous acid solution containing one or more inorganic acids and having a pH in the range from equal to or more than 1.8 to equal to or less than 4.0 to adjust the overall pH of the mixture, containing the pretreated lignocellulosic material and the aqueous acid solution, to a pH in the range from equal to or more than 3.0 to equal to or less than 4.5, more preferably to a pH in the range from equal to or more than 3.5 to equal to or less than 4.5, in the essential absence of an external base.

Step a), respectively step i) is also referred to herein as “pretreatment” or “pretreatment step”.

Preferably the lignocellulosic material is contacted in step a), respectively in step i), with the aqueous acid solution at a temperature equal to or more than 130° C., more preferably equal to or more than 140° C. and most preferably equal to or more than 150° C. The lignocellulosic material is contacted with the aqueous acid solution preferably at a temperature equal to or less than 200° C., more preferably equal to or less than 185° C. and most preferably equal to or less than 170° C.

Preferably the lignocellulosic material is contacted with the aqueous acid solution in step a), respectively in step i), during a reaction time equal to or more than 0.5 minute, more preferably equal to or more than 1 minute and most preferably equal to or more than 2 minutes. Preferably, the lignocellulosic material may be contacted with the aqueous acid solution in step a), respectively in step i), during a reaction time equal to or more than 5 minutes, or even equal to or more than 10 minutes. The reaction time may for example even be equal to or more than 30 minutes. For practical purposes the reaction time may be equal to or less than 4 hours, preferably equal to or less than 2 hours.

Preferably the lignocellulosic material is contacted in step a), respectively in step i), with the aqueous acid solution at a total pressure of equal to or more than 0.1 MegaPascal (1 bar), more preferably equal to or more than 0.2 MegaPascal (2 bar) and most preferably equal to or more than 0.3 MegaPascal (3 bar). The lignocellulosic material is contacted with the aqueous acid solution preferably at a total pressure of equal to or less than 5 MegaPascal (50 bar), more preferably equal to or less than 4 MegaPascal (40 bar). If desired the process according to the invention also allows for lower total pressures to be used, for example a total pressure of equal to or less than 0.3 MegaPascal (3 bar), or even equal to or less than 2.5 MegaPascal (2.5 bar).

In addition to the aqueous acid solution, preferably steam is supplied. Hence, in a preferred embodiment the lignocellulosic material is contacted with the aqueous acid solution and steam.

In the processes of the invention an aqueous acid solution, containing one or more inorganic acids and having a pH in the range from equal to or more than 1.8 to equal to or less than 4.0, is used. That is, the aqueous acid solution, contains one or more inorganic acids and the aqueous acid solution has a pH in the range from equal to or more than 1.8 to equal to or less than 4.0.

The aqueous acid solution may contain one or more acids. For example the aqueous acid solution may contain one or more inorganic acids and optionally one or more organic acids.

The one or more inorganic acids can be any type of inorganic acid known to be suitable in the pretreatment of lignocellulosic material. Preferably the one or more inorganic acid(s) comprise one or more inorganic acids chosen from the group consisting of sulphuric acid, sulphurous acid, hydrochloric acid, nitric acid, phosphorous acid, phosphoric acid and combinations thereof. In a preferred embodiment the aqueous acid solution is an aqueous acid solution of one or more inorganic acid(s) containing essentially no organic acids before being contacted with the lignocellulosic material feed.

In yet another preferred embodiment the aqueous acid solution is an aqueous acid solution containing one or more inorganic acid(s) and one or more organic acid(s). The one or more inorganic acid(s) are preferably chosen from the group listed above. The one or more organic acid(s) are preferably chosen from the group consisting of formic acid, acetic acid, citric acid, oxalic acid, levulinic acid and combinations thereof. In one embodiment one or more of the organic acid(s) may originate from the lignocellulosic material. For example, after use in step a) the aqueous acid solution may be at least partly retrieved and recycled for re-use as an aqueous acid solution containing one or more inorganic acid(s) and one or more organic acid(s).

In a more preferred embodiment the aqueous acid solution is an aqueous acid solution comprising a sulphur-containing acid. In a most preferred embodiment the aqueous acid solution is an aqueous acid solution of sulphuric acid. That is, in a most preferred embodiment the aqueous acid solution is an aqueous acid solution containing sulphuric acid. Preferably such an aqueous acid solution of sulphuric acid comprises in the range from equal to or more than 0.00001 wt %, more preferably equal to or more than 0.0001 wt % and most preferably equal to or more than 0.001 wt % sulphuric acid to equal to or less than 10 wt %, more preferably equal to or less than 1.0 wt %, even more preferably equal to or less than 0.5 wt %, still more preferably equal to or less than 0.1 wt %, and most preferably equal to or less than 0.08 wt % sulphuric acid, based on the total weight of the aqueous acid solution. For example the aqueous acid solution preferably comprises in the range from equal to or more than 0.00001 wt % to equal to or less than 0.1 wt % sulphuric acid, based on the total weight of the aqueous acid solution; more preferably in the range from equal to or more than 0.00001 wt % to equal to or less than 0.08 wt % sulphuric acid, based on the total weight of the aqueous acid solution. Such an aqueous acid solution of sulphuric acid may contain one or more additional acids. Preferably, however, the aqueous acid solution of sulphuric acid consists essentially of water and sulphuric acid.

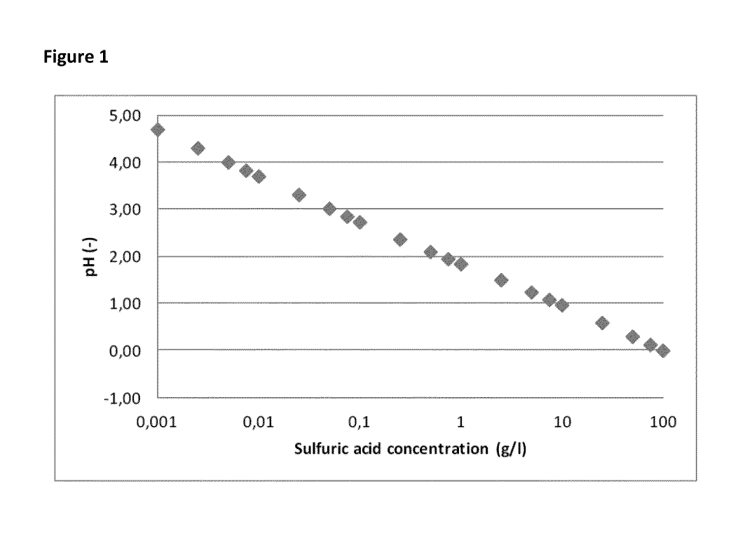

If an acid other than sulphuric acid is used or if a mixture of acids is used, such an acid or mixture of acids is preferably used in such a concentration that a pH is obtained that corresponds with the pH as obtained with the concentration of sulphuric acid as listed above. Examples of corresponding pH for specific sulphuric acid concentrations are summarized in Table I and in FIG. 1.

TABLE 1
Sulphuric acid concentration and corresponding pH

| H2SO4 | | pH |
|---|---|---|
| g/l | (wt %) | (—) |
| 0.001 | 0.0001 | 4.69 |
| 0.0025 | 0.00025 | 4.29 |
| 0.005 | 0.0005 | 3.99 |
| 0.0075 | 0.00075 | 3.82 |
| 0.01 | 0.001 | 3.69 |
| 0.025 | 0.0025 | 3.30 |
| 0.05 | 0.005 | 3.01 |
| 0.075 | 0.0075 | 2.84 |
| 0.1 | 0.01 | 2.72 |
| 0.25 | 0.025 | 2.35 |
| 0.5 | 0.05 | 2.09 |
| 0.75 | 0.075 | 1.94 |
| 1 | 0.1 | 1.83 |
| 2.5 | 0.25 | 1.49 |
| 5 | 0.5 | 1.22 |
| 7.5 | 0.75 | 1.07 |
| 10 | 1 | 0.95 |
| 25 | 2.5 | 0.57 |
| 50 | 5 | 0.28 |
| 75 | 7.5 | 0.11 |
| 100 | 10 | −0.01 |

The pH of the aqueous acid solution before reaction in step a) respectively before reaction in step i) is also referred to herein as pre-reaction pH. To prepare the aqueous acid solution of an inorganic acid, the inorganic acid can be diluted with water until the specified pH is reached. The pH of the aqueous acid solution of the inorganic acid (that is the pH before reaction) is preferably equal to or more than 1.9, more preferably equal to or more than 2.0, even more preferably equal to or more than 2.1, still more preferably equal to or more than 2.2, even still more preferably equal to or more than 2.3 and most preferably equal to or more than 2.4. For practical purposes the pH of the aqueous acid solution of the inorganic acid is preferably equal to or less than 3.9, more preferably equal to or less than 3.8, even more preferably equal to or less than 3.7, still more preferably equal to or less than 3.6, even still more preferably equal to or less than 3.5 and most preferably equal to or less than 3.4.

Preferably the weight ratio of lignocellulosic material (on a dry basis) to aqueous acid solution (also referred to as lignocellulosic material:aqueous acid solution ratio) in step a), respectively in step i) lies in the range from equal to or more than 1:1 to equal to or less than 1:15; more preferably in the range from equal to or more than 1:1 to equal to or less than 1:10; most preferably in the range from equal to or more than 1:2 to equal to or less than 1:4.

Preferably the mixture produced in step a) is a slurry of pretreated lignocellulosic material and aqueous acid solution. Analogously the mixture produced in step i) is preferably a slurry of pretreated lignocellulosic material, aqueous acid solution and one or more, preferably dissolved, calcium salts. This slurry preferably has a solids content in the range from equal to or more than 3 wt % to equal to or less than 50 wt %, more preferably in the range from equal to or more than 10 wt % to equal to or less than 50 wt % and most preferably equal to or more than 20 wt % to equal to or less than 50 wt %, based on the total weight of slurry.

The aqueous acid solution can be added in a sufficient amount and concentration to adjust the overall pH of the mixture of the lignocellulosic material and the aqueous acid solution to an overall pH in the range from equal to or more than 3.0 to equal to or less than 4.5. For example, the aqueous acid solution can be added in a sufficient amount and concentration to adjust the overall pH of the mixture containing the pretreated lignocellulosic material, the aqueous acid and any one or more, preferably dissolved, calcium salts to an overall pH in the range from equal to or more than 3.0 to equal to or less than 4.5.

By an overall pH is herein understood the pH of the mixture containing pretreated lignocellulosic material and aqueous acid solution obtained post-reaction. Suitably this can also be referred to as the pH obtained after step a) respectively after step i) has been finalized.

The overall pH is herein also referred to as final pH or post-reaction pH.

Preferably the overall pH of the mixture of pretreated lignocellulosic material and aqueous acid solution in step a), suitably of the mixture of pretreated lignocellulosic material, aqueous acid solution, and one or more, preferably dissolved, calcium salts in step i) is equal to or more than 3.1, more preferably equal to or more than 3.2, even more preferably equal to or more than 3.3, still more preferably equal to or more than 3.4, even still more preferably equal to or more than 3.5 and most preferably equal to or more than 3.6. Preferably the overall pH is equal to or less than 4.4, more preferably equal to or less than 4.3, even more preferably equal to or less than 4.2, still more preferably equal to or less than 4.1 and most preferably equal to or less than 4.0.

As explained above, the overall pH may be reached by contacting the aqueous acid solution and the lignocellulosic material, without the necessity of adding an external base. That is, step a) respectively step i) can be carried out in the essential absence of an external base. Hence in a preferred embodiment step a) respectively step i), is carried out without the essential addition of an external base. By an external base is herein understood a basic compound that did not originate from the lignocellulosic material itself.

As explained above, step a) respectively step i) may include leaching of basic compounds from the lignocellulosic material during the reaction to adjust the overall pH to a pH in the range from equal to or more than 3.0 to equal to or less than 4.5, more preferably to a pH in the range from equal to or more than 3.5 to equal to or less than 4.5.

Step a), respectively step i) may be carried out in a batchwise, semi-batchwise or continuous manner. Preferably step a), respectively step i), is carried out in a continuous manner. In step a), respectively in step i), the lignocellulosic material is preferably contacted with the aqueous acid solution in a reactor. Any type of reactor known to be suitable for the pretreatment of lignocellulosic material may be used in step a), respectively in step i). For example step a) respectively step i) may be carried out in one or more plug flow reactor(s), one or more continuous stirred tank reactor(s) or a combination thereof. The one or more reactors may include one or more essentially horizontally arranged reactor(s) and/or one or more essentially vertically arranged reactor(s). Preferably at least part of step (a), respectively at least part of step i), is carried out in an essentially horizontally arranged reactor.

In a preferred embodiment at least part of step (a), respectively at least part of step i), is carried out in an essentially tubular shaped reactor (also referred to as tube reactor or tubular reactor). Preferably such a tubular reactor is an essentially horizontally arranged tubular reactor. The tubular reactor may be a compartmentalized tubular reactor, for example a tubular reactor comprising a screw or other mechanical displacement device.

In a further preferred embodiment at least part of step (a), respectively at least part of step i), is carried out in a reactor essentially operated at plug flow (also referred to as plug flow reactor). Without wishing to be bound by any kind of theory it is believed that when operated at plug flow, the residence time in the reactor is essentially the same for all elements in the reaction mixture. A more extensive explanation of plug flow can be found in chapter 13 of the handbook by O. Levenspiel, titled “Chemical Reaction Engineering”, 3th Edition, 1999, published by John Wiley & Sons, New York, herein incorporated by reference.

A plug flow may for example be created in a tubular reactor, and preferably step a), respectively step i), is carried out in a tubular reactor operated at plugflow. It may also be created in a compartmentalized tubular reactor or in another reactor or series of reactors having multiple compartments being transported forward, where preferably each of these compartments are essentially completely mixed. An example of a compartmentalized tubular reactor operated at plug flow may be a tubular reactor comprising a screw.

The use of a plug flow reactor may be advantageous to avoid so-called overcooking and/or undercooking during step a), respectively during step i).

The reactor in step a), respectively in step i), may conveniently comprise a mechanical displacement device such as for example a device chosen from the group of conveyors, pumps, screws, plungers, moving belts, moving chains and/or combinations thereof.

Step a), respectively in step i), may suitably comprise mixing of the lignocellulosic material with the aqueous acid solution.

The lignocellulosic material and aqueous acid solution and optionally steam may be premixed before entering a reactor. In a preferred embodiment the lignocellulosic material and the aqueous acid solution are premixed before entering a reactor to form a premixed composition and subsequently the premixed composition of lignocellulosic material and aqueous acid solution is contacted with steam in the reactor. Conveniently the steam may be used to regulate pressure and/or temperature in the reactor.

In an especially preferred embodiment the lignocellulosic material is pre-soaked in the aqueous acid solution at a pressure of about 1 bar absolute and a temperature in the range from 18° C. to 100° C., before being fed into a reactor in step a), respectively in step i). Conveniently the pre-soaking may be carried out in a stirred vessel, where preferably the lignocellulosic material and the aqueous acid solution are mixed. Such pre-soaking advantageously may allow for a smaller shift in pH during the reaction in the reactor and may allow a better process control and more robust operation. This pre-soaked lignocellulosic material preferably has a solids content of equal to or more than 3 wt %, more preferably equal to or more than 10 wt %, even more preferably equal to or less than 20 wt % and most preferably equal to or more than 30 wt %, based on the total weight of pre-soaked lignocellulosic material. For practical purposes the solid content is preferably equal to or less than 90 wt %, more preferably equal to or less than 80 wt %, based on the total weight of pre-soaked lignocellulosic material.

The residence time in a reactor in step a), respectively a reactor in step i), may vary widely. Preferably the residence time is equal to or more than 0.5 minute, more preferably equal to or more than 1 minute, still more preferably equal to or more than 2 minutes. Even more preferably the residence time is equal to or more than 10 minutes and most preferably the residence time is equal to or more than 15 minutes. For practical purposes the residence time is preferably equal to or less than 4 hours, more preferably equal to or less than 2 hours, still more preferably equal to or less than 1 hour, even more preferably equal to or less than 30 minutes and most preferably equal to or less than 20 minutes.

In step a) a mixture is produced, containing pretreated lignocellulosic material and aqueous acid solution, having an overall pH in the range from equal to or more than 3.0 to equal to or less than 4.5. That is, the mixture contains pretreated lignocellulosic material and aqueous acid solution and the mixture has an overall pH in the range from equal to or more than 3.0 to equal to or less than 4.5.

Preferably the pretreated lignocellulosic material contains a total amount of calcium equal to or more than 50 wt %, more preferably equal to or more than 70 wt %, still more preferably equal to or more than 80 wt % and most preferably equal to or more than 90 wt % of the total amount of calcium in the lignocellulosic material used as a feed to step a) respectively as a feed to step i). For practical purposes the pretreated lignocellulosic material may contain a total amount of calcium equal to or less than 100 wt %, more preferably equal to or more than 99 wt % of the total amount of calcium in the lignocellulosic material used as a feed to step a), respectively as a feed to step i).

The pretreated lignocellulosic material preferably contains equal to or more than 10 ppmw (mg/kg), preferably equal to or more than 50 ppmw, more preferably equal to or more than 100 ppmw, still more preferably equal to or more than 500 ppmw and most preferably equal to or more than 1000 ppmw of an alkali metal and/or an alkaline earth metal and/or a mixture thereof bound to the lignocellulosic material, based on the total weight of pretreated lignocellulosic material on a dry basis. The alkali metal and/or alkaline earth metal and/or a mixture thereof preferably comprise calcium. Hence, the pretreated lignocellulosic material preferably contains equal to or more than 10 ppmw (mg/kg), preferably equal to or more than 50 ppmw, more preferably equal to or more than 100 ppmw, still more preferably equal to or more than 500 ppmw and most preferably equal to or more than 1000 ppmw of calcium bound to the lignocellulosic material, based on the total weight of pretreated lignocellulosic material on a dry basis. By a dry basis is understood that first water is removed from the lignocellulosic material before the weight percentage is calculated. The content in ppmw is further calculated as an elemental weight percentage.

The calcium salts, alkali metal salts and/or alkaline earth metal salts or any salts referred to herein as “insoluble salts” can be present in the mixture produced in step a) as solid salts or dissolved salts, and are preferably present as dissolved salts. By a dissolved salt is herein preferably understood a salt that is dissolved in a solution. Such a solution may for example be a solution in water or a solution in the aqueous acid solution. Dissolved salt may also be referred to as electrolytes. For example, the one or more dissolved calcium salts in step i) may comprise an aqueous solution of calcium electrolytes. Hence, the mixture produced in step i) may for example be a mixture containing pretreated lignocellulosic material and an aqueous solution of dissolved calcium electrolytes.

Before providing the mixture produced in step a) to a subsequent step, respectively before providing any mixture produced in step i) to a subsequent step, part of the water may be removed from it.

In one embodiment at least part of the water is removed from the mixture produced in step a) before providing it to step b), respectively from the mixture produced in step i) before providing it to step ii). For example, the mixture produced in step a), respectively the mixture produced in step i) may be partially or wholly depressurized in one or more flashing steps. This may advantageously reduce the volume of the equipment more downstream. However, if part of the water is removed from the mixture, it is preferred to maintain the pH within the ranges as mentioned above for the overall pH of the mixture. Preferably no external base is added during such water removal.

In another embodiment essentially no water is removed between steps a) and b), respectively between steps i) and ii). This allows one to ensure that the overall pH of mixture produced in step a), respectively the pH of the mixture produced in step i), is maintained and that the pH does not, for example, decrease below the lower pH threshold.

In another embodiment the pretreated lignocellulosic material is washed before providing it to a subsequent step. For example, the pretreated lignocellulosic material may be washed with water. The pretreated lignocellulosic material may be washed in one or more washing cycles and is preferably washed in one or more water-washing cycles. For example, the pretreated lignocellulosic material obtained in step a), respectively in step i), may optionally be washed with water in a washing step before forwarding the pretreated lignocellulosic material to any subsequent step.

In one embodiment the process of the invention comprises a step b) of contacting at least part of the mixture produced in step a) with a base to produce a neutralized mixture containing neutralized pretreated lignocellulosic material and one or more insoluble salts.

In step b) at least part of the mixture produced in step a) is contacted with a base to produce a neutralized mixture containing neutralized pretreated lignocellulosic material and one or more insoluble salts.

In step b) the pH of the mixture or part thereof is preferably increased to a pH of equal to or more than 4.0, more preferably equal to or more than 4.4, still more preferably equal to or more than 4.5 and preferably to equal to or less than 7.0, more preferably equal to or less than 6.0. For example the pH may be increased to a pH in the range of equal to or more than 4.0 to equal to or less than 7.0, preferably to a pH in the range of equal to or more than 4.5 to equal to or less than 6.0.

In step b) the mixture or part thereof may be contacted with one or more bases. These one or more bases may include for example solid bases, dissolved bases and/or a combination thereof. Preferably the base used in step b) comprises one or more basic compounds that are soluble in water under standard conditions of 1 bar atmosphere and 20° C.

By a base or basic compound is herein understood a species that, when dissolved in water, gives a solution with a pH that is more than 7. The base may comprise any organic and/or inorganic basic compound. Preferably, however, the base comprises an inorganic basic compound. For example the base may be chosen from the group consisting of ammonia, ammonium hydroxide, potassium hydroxide, sodium hydroxide, calcium hydroxide, magnesium hydroxide, potassium carbonate, sodium carbonate, potassium bicarbonate, sodium bicarbonate and combinations thereof. These bases may be present in solid or dissolved form. Preferably the base in step b) is sodium hydroxide, potassium hydroxide, ammonia and/or ammonium hydroxide. The base is preferably added as an aqueous basic solution of the basic compound. The base or basic compound can also suitably be added in the form of a pH buffer, for example sodium carbonate and citric acid may suitably be used to form a sodium citrate buffer.

Step b) produces a neutralized mixture containing neutralized pretreated lignocellulosic material and one or more insoluble salts. By a neutralized mixture is herein understood a mixture having a higher pH than the mixture produced in step a). By a neutralized pretreated lignocellulosic material is herein understood a pretreated lignocellulosic material having a higher pH than the pretreated lignocellulosic material produced in step a).

Preferably the neutralized mixture has a pH in the range from equal to or more than 4.0 to equal to or less than 7.0, more preferably in the range from equal to or more than 4.5 to equal to or less than 6.0.

Preferably the neutralized pretreated lignocellulosic material has a pH in the range from equal to or more than 4.0 to equal to or less than 7.0, more preferably in the range from equal to or more than 4.5 to equal to or less than 6.0.

As explained above, the amount of insoluble salts formed has been greatly reduced by the process of the invention. In a preferred embodiment the neutralized mixture contains equal to or less than 9.0 milligram, more preferably equal to or less than 5.0 milligram, even more preferably equal to or less than 2.0 milligram and most preferably equal to or less than 1.5 milligram of insoluble salts per gram of neutralized pretreated lignocellulosic material calculated on a dry basis. For practical purposes the neutralized mixture may contain equal to or more than 0.01 milligram, more preferably equal to or less than 0.1 milligram of insoluble salts per gram of neutralized pretreated lignocellulosic material calculated on a dry basis. For example the neutralized mixture may contain in the range from equal to or more than 0.01 milligram to equal to or less than 5.0 milligram of insoluble salts per gram of neutralized pretreated lignocellulosic material, calculated on a dry basis.

Preferably the insoluble salts are salts of one or more alkali metal(s) and/or alkaline earth metal(s) that are essentially not soluble in the neutralized mixture produced in step b). Preferably the insoluble salts are calcium salts. Hence, in a preferred embodiment the neutralized mixture contains calcium salts that are not soluble in the neutralized mixture. In a special embodiment the insoluble salts are salts selected from the group consisting of calcium sulphate, calcium bisulphate, calcium sulphite, calcium bisulphite, calcium carbonate, calcium acetate and mixtures thereof.

Preferably the neutralized mixture contains in the range from equal to or more than 0.01 milligram to equal to or less than 5.0 milligram, more preferably equal to or less than 2.0 milligram, of calcium salts, per gram of neutralized pretreated lignocellulosic material calculated on a dry basis.

In a preferred embodiment the one or more insoluble salts produced in step b) are one or more salts selected from the group consisting of calcium sulphate, calcium bisulphate, calcium sulphite, calcium bisulphite, calcium carbonate, calcium acetate and mixtures thereof. Most preferably the one or more insoluble salts are selected from the group consisting of calcium sulphate, calcium bisulphate and mixtures thereof. Hence, in a preferred embodiment step b) comprises contacting at least part of the mixture produced in step a) with a base to produce a neutralized mixture containing neutralized pretreated lignocellulosic material and one or more salts selected from the group consisting of calcium sulphate, calcium bisulphate, calcium sulphite, calcium bisulphite, calcium carbonate, calcium acetate and mixtures thereof. The sulphate salts and bisulphate salts may for example form when sulphuric acid is used as an inorganic acid in step a) or if the base used in step b) comprises a sulphate or bisulphate salt.

In addition to the neutralized pretreated lignocellulosic material and the insoluble salts, the neutralized mixture may also contain for example lignin and xylose.

If desired, water may be removed from the neutralized mixture produced in step b). For example, the neutralized mixture produced in step b) may be partially or wholly depressurized in one or more flashing steps.

If the mixture produced in step a) or the neutralized mixture produced in step b) is partially or wholly depressurized in one or more flashing steps, the pressure is preferably reduced to a pressure in the range of equal to or more than 0.1 MegaPascal (1 bar) to equal to or less than 1 MegaPascal (10 bar), more preferably equal to or less than 0.5 MegaPascal (5 bar), most preferably equal to or less than 0.3 MegaPascal (3 bar). One or more flashing steps may be used. Preferably 2 to 8 flashing steps are used, more preferably 2 to 6 flashing steps are used. Such partial or wholly depressurization may for example be carried out as described in WO2006/128304.

In another embodiment the neutralized pretreated lignocellulosic material is washed before providing it to a subsequent step. For example, the neutralized pretreated lignocellulosic material may be washed with water. The neutralized pretreated lignocellulosic material may be washed in one or more washing cycles and is preferably washed in one or more water-washing cycles. For example, the neutralized pretreated lignocellulosic material obtained in step b) may optionally be washed with water in a washing step before forwarding the neutralized pretreated lignocellulosic material to any subsequent step.

The neutralized pretreated lignocellulosic material produced in step b) can advantageously be used in any process that converts a lignocellulosic material into one or more bio-fuel(s) and/or one or more bio-chemical(s).

For example the neutralized pretreated lignocellulosic material can be converted to one or more hydrocarbons, for example hydrocarbons comprising in the range from 6 to 20 carbon atoms. Such hydrocarbons can for example be useful as a component in a gasoline and/or diesel fuel or in a lubricant.

The neutralized pretreated lignocellulosic material may also conveniently be converted to one or more alkanol(s), for example ethanol and/or butanol.

In a preferred embodiment the, preferably neutralized, pretreated lignocellulosic material is converted in a process that comprises hydrolyzing at least part of the neutralized pretreated lignocellulosic material to produce a hydrolysis product. Preferably the hydrolysis of at least part of the, preferably neutralized, pretreated lignocellulosic material comprises enzymatic hydrolysis. For example the process may comprise hydrolyzing at least part of the neutralized pretreated lignocellulosic material produced in step b) to produce a hydrolysis product, whereafter the hydrolysis product is preferably converted into one or more bio-fuel(s) and/or one or more bio-chemical(s). Preferences for such a hydrolysis are described in more detail below.

The present invention therefore also provides a process for the production of one or more alkanol(s) comprising the steps a) and b) as described herein above, followed by:

a step c) comprising hydrolyzing at least part of the neutralized pretreated lignocellulosic material produced in step b) to produce a hydrolysis product; and
a step d) comprising fermenting at least part of the hydrolysis product produced in step c) to produce a fermentation broth comprising the one or more alkanol(s).

Preferably such steps c) and d) are followed by:

an optional step e) comprising retrieving the one or more alkanols from the fermentation broth produced in step d).

The invention further provides a process for the production of a fuel comprising the steps of:

a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having an overall pH in the range from equal to or more than 3.0 to equal to or less than 4.5, containing pretreated lignocellulosic material and aqueous acid solution;

b) contacting at least part of the mixture produced in step a) with a base to produce a neutralized mixture containing neutralized pretreated lignocellulosic material and one or more insoluble salts;

c) hydrolyzing at least part of the neutralized pretreated lignocellulosic material produced in step b) to produce a hydrolysis product;

d) fermenting at least part of the hydrolysis product produced in step c) to produce a fermentation broth comprising the one or more alkanol(s);

optional step e) comprising retrieving the one or more alkanols from the fermentation broth produced in step d); and

further comprising an additional step of blending the one or more alkanols produced in step d) and/or e) with one or more other fuel components to produce a fuel.

Preferences for steps a) and b) are described in more detail above. Preferences for preferred steps c), d) and/or e) are described in more detail below.

In preferred step c) at least part of the neutralized pretreated lignocellulosic material produced in step b) is hydrolyzed to produce a hydrolysis product.

The hydrolysis may be carried out in any manner known to the skilled person in the art to be suitable for the hydrolysis of a lignocellulosic material. Preferably the neutralized pretreated lignocellulosic material produced in step b) is hydrolyzed in step c) by enzymatic hydrolysis. In an especially preferred embodiment the hydrolysis comprises hydrolyzing the neutralized pretreated lignocellulosic material with the help of one or more cellulase enzymes. A cellulase enzyme (also sometimes referred to as “cellulase”) can catalyse the hydrolysis of cellulose present in the neutralized pretreated lignocellulosic material. The cellulase enzyme may be any cellulase enzyme known to the skilled person to be suitable for hydrolysis of cellulose. Examples of suitable cellulase enzymes include cellulase enzymes obtained from fungi of the genera Aspergillus, Humicola and Trichoderma and/or Myceliophthora and from the bacteria of the genera Bacillus and Thermobifida.

Examples of the cellulase enzymes include cellobiohydrolases (CBH's), endoglucanases (EG's), beta-glucosidases and mixtures thereof. In addition to cellulase enzymes, hemicellulase enzymes, esterase enzymes and swollenins may be present. The cellulase enzyme dosage may for example be in the range from 5.0 to 100.0 Filter Paper Units (FPU or IU) per gram of cellulose. The FPU is a standard measurement and is defined and measured according to Ghose (1987, Pure and Appl. Chem. 59: pages 257-268).

Preferably any enzymatic hydrolysis in step c) is carried out at a temperature of equal to or more than 15° C., more preferably equal to or more than 20° C. and most preferably equal to or more than 25° C. whilst the temperature is preferably equal to or less than 50° C., more preferably equal to or less than 40° C. and most preferably equal to or less than 35° C. Hence, preferably the enzymatic hydrolysis is carried out at a temperature in the range from equal to or more than 15° C. to equal to or less than 40° C.

Preferably the enzymatic hydrolysis is carried out for a reaction time equal to or more than 1 hour, more preferably equal to or more than 5 hours, even more preferably equal to or more than 10 hours. And preferably the enzymatic hydrolysis is carried out for a reaction time equal to or less than 300 hours, more preferably equal to or less than 200 hours, most preferably equal to or less than 100 hours. Hence, preferably the enzymatic hydrolysis is carried out for a reaction time in the range from equal to or more than 1 hour to equal to or less than 200 hours.

By hydrolysis of the neutralized pretreated lignocellulosic material containing cellulose a hydrolysis product is produced. The hydrolysis product may contain one or more sugars. The sugars may comprise for example monosaccharides and disaccharides. For example the hydrolysis product may contain glucose, xylose, galactose, mannose, arabinose, fructose, rhamnose and/or mixtures thereof. In addition to the hydrolysis product the effluent from step c) may optionally contain lignin and any unconverted pretreated lignocellulosic material.

Where step c) produces an effluent containing a liquid hydrolysis product and one or more solids, the process according to the invention may optionally include an additional step after step c) and before step d) where the liquid hydrolysis product is separated from such solids by means of a liquid/solid separation. Examples of solids that may be present in the effluent of step c) include lignin and/or unconverted pretreated lignocellulosic material. For example if the effluent of step c) comprises a slurry of an aqueous solution of sugars with solid lignin and solid unconverted pretreated lignocellulosic material, a solid-liquid separation may be carried out to separate the hydrolysis product from such solid lignin and/or any solid unconverted pretreated lignocellulosic material. The recovered solids may be burned to provide energy.

In step d) at least part of the hydrolysis product produced in step c) can be fermented to produce a fermentation broth.

The fermentation in step d) may for example be carried out with the help of a microorganism. The microorganism may be any kind of microorganism known to be capable of fermenting part or whole of the hydrolysis product. For example, it may be a microorganism capable of fermenting part or whole of the hydrolysis product to a fermentation broth containing ethanol and/or butanol. Preferably the microorganism is chosen from the group consisting of Saccharomyces spp., Saccharomyces cerevisiae, Escherichia, Zymomonas, Candida, Pichia, Streptomyces, Bacillus, Lactobacillus, Clostridium and mixtures thereof.

Preferably the fermentation in step d) is carried out at a temperature of equal to or more than 15° C., more preferably equal to or more than 20° C. and most preferably equal to or more than 25° C. whilst the temperature is preferably equal to or less than 50° C., more preferably equal to or less than 40° C. and most preferably equal to or less than 35° C.

Preferably the fermentation in step d) is carried out at a pH in the range from equal to or more than 3.0 and equal to or less than 6.0, more preferably in the range from equal to or more than 4.0 to equal to or less than 6.0. If desired one or more additional nutrients for the microorganism may be added to step d), such as for example yeast extract, specific amino acids, phosphate, nitrogen sources, salts, trace elements and vitamins.

The fermentation may be carried out in batch, continuous or fed-batch mode with or without agitation. The fermentation may be carried out in one or more reactors, preferably in a series of 1 to 6 fermentation reactors. Preferably the fermentation is carried out in one or more mechanically stirred reactors. The fermentation microorganisms may be recycled back to the fermentation reactor. Or they may for example be sent to distillation without recycle. In one embodiment the hydrolyzing of step c) and the fermentation of step d) are carried out simultaneously in the same reactor. It is, however, most preferred to carry out the hydrolyzing of step c) and the fermentation of step d) separately to allow for optimal temperatures for each step.

The fermentation broth generated in step d) may contain one or more alkanols. Preferably the fermentation broth contains ethanol and/or butanol. Most preferably the fermentation broth is a fermentation broth containing ethanol. In addition the fermentation broth may contain water and/or solids. Examples of solids that may be present in the fermentation broth include unconverted pretreated lignocellulosic material, lignin and/or any solid components added during fermentation. In addition microorganisms may be present in the fermentation broth depending on whether or not such microorganisms have been recycled during step d).

Where step d) produces a fermentation broth containing a liquid and one or more solids, the process according to the invention may optionally include an additional step after step d) where solids are removed from the fermentation broth by means of a liquid/solid separation.

In optional step e) the one or more alkanols are retrieved from the fermentation broth produced in step d).

Preferably step e) comprises distillation of the fermentation broth to produce one or more distillation fraction(s) comprising the one or more alkanol(s), for example a distillation fraction comprising ethanol and/or a distillation fraction comprising butanol and/or a distillation fraction comprising ethanol and butanol. A distillation in step e) may comprise one or more distillation columns. The fermentation broth is preferably first degassed to remove carbon dioxide before distillation. In addition to one or more distillation fraction(s) containing one or more alkanol(s), the distillation of the fermentation broth may generate one or more residue fraction(s). In one embodiment such one or more residue fraction(s) contain(s) one or more insoluble salts.

The one or more alkanol(s), for example the butanol and/or ethanol, may advantageously be blended with one or more other components to produce a biofuel or a biochemical. Examples of one or more other components with which the one or more alkanol(s) may be blended include anti-oxidants, corrosion inhibitors, ashless detergents, dehazers, dyes, lubricity improvers and/or mineral fuel components and/or other fuel components, such as for example so-called Fischer-Tropsch derived fuel components or other renewable fuel components.

The present invention therefore also provides a process to for the production of a fuel comprising steps a), b), c), d) and optionally e) as described herein above and further comprising an additional step of blending the one or more alkanols produced in step d) and/or e) with one or more other fuel components to produce a fuel.

The processes according to the invention further preferably comprise a desalting step. This desalting step may for example comprise removing and/or retrieving one or more insoluble salts produced in step b).

The desalting step may comprise desalting of the mixture produced in step a), the neutralized mixture produced in step b) and/or the neutralized pretreated lignocellulosic material produced in step b) and/or the hydrolysis product produced in step c) and/or the fermentation broth produced in step d) and/or one or more distillate fraction(s) and/or one or more residue fraction(s) obtained in optional step e).

In one embodiment the desalting step comprises desalting of the neutralized mixture produced in step b) and/or the neutralized pretreated lignocellulosic material produced in step b).

In another embodiment, however, insoluble salts present in the neutralized mixture and/or the neutralized pretreated lignocellulosic material produced in step b) can be carried over in subsequent steps and the desalting step comprises desalting of the product of such a subsequent step. For example the insoluble salts can be carried over through the hydrolysis in step c), the fermentation in step d) and/or the optional distillation in step e). In this embodiment the insoluble salts can be removed and/or retrieved from the hydrolysis product produced in step c) and/or the fermentation broth produced in step d) and/or the one or more distillate fraction(s) and/or the one or more residue fraction(s) produced in optional step e). In this later case, the desalting step may also be referred to as step f).

In a first embodiment the desalting step comprises electrodialysis of a product containing the one or more insoluble salts to produce a concentrated salt solution; and insoluble salts are removed and/or retrieved from the concentrated salt solution by means of crystallization. Examples of products containing the one or more insoluble salts that may be electrodialysed include the neutralized mixture produced in step b) and/or the hydrolysis product produced in step c) and/or the fermentation broth produced in step d) and/or one or more distillation fraction(s) produced in optional step e).

In another embodiment the desalting step comprises anaerobic fermentation of a product containing the one or more insoluble salts to produce a desalting residue containing the insoluble salts; and recovering the insoluble salts from the residue.

Examples of products containing the one or more insoluble salts that are suitable for anaerobic fermentation include the neutralized mixture produced in step b) and/or the neutralized pretreated lignocellulosic material produced in step b) and/or the hydrolysis product produced in step c) and/or the fermentation broth produced in step d) and/or one or more distillate fraction(s) and/or one or more residue fraction(s) obtained in optional step e).

In a still further embodiment the desalting step comprises contacting of a product containing the one or more insoluble salts with one or more ion-exchange resins to produce a concentrated salt solution; and removing and/or retrieving the insoluble salts from the concentrated salt solution by means of crystallization.

Examples of products containing the one or more insoluble salts that are suitable for contacting with ion-exchange resins include the neutralized mixture produced in step b) and/or the hydrolysis product produced in step c) and/or the fermentation broth produced in step d) and/or one or more distillate fraction(s) and/or one or more residue fraction(s) obtained in optional step e).

In one preferred embodiment the desalting step comprises:

(I) removing water from the fermentation broth produced in step d) and/or one or more distillate fraction(s) and/or one or more residue fraction(s) obtained in optional step e) by means of evaporation to produce a concentrated product;
(II) burning the concentrated product produced in step (I) to produce ashes;
(III) removing and/or retrieving alkali metal salts and/or alkali metal earth salts from the ashes.

Preferably the concentrated product obtained in step (I) comprises in the range from 50 to 90 wt % solids and in the range from 10 to 50 wt % water, based on the total weight of the concentrated product.

As indicated above, the invention also provides a process comprising

i) contacting the calcium-containing lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having a pH in the range from equal to or more than 3.0 to equal to or less than 4.5, containing pretreated lignocellulosic material and one or more, preferably dissolved, calcium salts;

ii) retrieving at least part of the one or more calcium salts.

The pH of the mixture containing pretreated lignocellulosic material, suitably aqueous acid solution and one or more, preferably dissolved, calcium salts produced in step i) may hereafter also be referred to as overall pH, post-reaction pH or final pH.

Preferences for step i) are as described above for step a). In addition, step i) may preferably further include steps identical to steps b), c), d) and/or e) as described herein above. Preferences for step ii) are as described above for the desalting step.

As indicated above, in a specially preferred embodiment the lignocellulosic material is a calcium-containing lignocellulosic material. The present invention therefore further provides a process for processing a calcium-containing lignocellulosic material comprising the steps of
a) contacting the calcium-containing lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having an overall pH in the range from equal to or more than 3.0 to equal to or less than 4.5, containing pretreated lignocellulosic material and one or more, preferably dissolved, calcium salts;
b) contacting at least part of the mixture produced in step a) with a base to produce a neutralized mixture containing neutralized pretreated lignocellulosic material and one or more solid calcium salts.

The neutralized pretreated lignocellulosic material may conveniently be converted to an alkanol such as ethanol and/or butanol and hence the current invention also provides a process for the production of one or more alkanol(s) comprising the steps of
a) contacting a calcium-containing lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture having an overall pH in the range from equal to or more than 3.0 to equal to or less than 4.5, containing pretreated lignocellulosic material and one or more, preferably dissolved, calcium salts;
b) optionally contacting at least part of the mixture produced in step a) with a base to produce a neutralized mixture containing neutralized pretreated lignocellulosic material and one or more solid calcium salts;
c) hydrolyzing at least part of the pretreated lignocellulosic material produced in step a) and/or at least part of the neutralized pretreated lignocellulosic material produced in step b) to produce a hydrolysis product;
d) fermenting at least part of the hydrolysis product produced in step c) to produce a fermentation broth comprising the one or more alkanol(s).
   Preferences for these steps a), b), c) and/or d) are as described above for steps a), b), c) and/or d). The above processes may further be supplemented by a step e), similar to previously described step e). In addition the above processes may comprise a desalting step as herein described before.

### EXAMPLES

### Examples 1 to 7 and Comparative Examples A to K

### Pretreatment Step

For examples 1 to 7 and comparative examples A to K, an aqueous acid solution was prepared using the acid as indicated in table 2 with a pre-reaction pH as indicated in table 2. The aqueous acid solution and a lignocellulosic material feed as listed in table 2 were weighted into an autoclave equipped with a stirrer. The wheat straw used as a lignocellulosic material feed contained 1695 ppmw calcium, 643 ppmw magnesium, 9020 ppmw potassium, 312 ppmw phosphorus and 75 ppmw sodium, as determined via inductively coupled plasma-atomic emission spectroscopy (ICP-AES). The Birchwood used as a lignocellulosic material feed contained 4365 ppmw calcium, 357 ppmw magnesium, 1350 ppmw potassium, 512 ppmw phosphorus and 21 ppmw sodium, as determined via inductively coupled plasma-atomic emission spectroscopy (ICP-AES). A ratio of 1:10 of the lignocellulosic material to aqueous acid solution was used in each case. In all cases except comparative examples J and K, 10 gram of lignocellulosic material was weighed into the autoclave followed by 100 gram of the aqueous acid solution. In the comparative examples J and K, 15 gram of wheat straw and 150 gram of aqueous acid solution were used. The autoclave was closed and the stirrer turned on at 300 rpm. The autoclave was heated with a heater to the required reaction temperature as listed in table 2, taking approximately 27 minutes to reach 150° C. and 32 minutes to reach 170° C. Reaction time as listed in table 2 was measured starting at the point in time (t=0) when the reaction temperature as listed in table 2 was reached. After the indicated reaction time, the heater was removed and the autoclave was cooled in water. Once cooled, a mixture of aqueous acid solution and pretreated lignocellulosic material was retrieved from the autoclave and poured into a Buchner flask with a P3 or P4 filter, generating a liquid filtrate and a solid residue. The solid residue contains pretreated lignocellulosic material. The pH of the liquid filtrate was measured and listed in table 2 as the post-reaction pH. The pre-reaction pH and post-reaction pH were determined using a Mettler Toledo Seven Multi pH meter.

Subsequently the residue was washed twice with 100 ml demineralized water.

The degree of liquefaction was calculated as follows:
Drying the lignocellulosic material used as a feed over-night (about 16 hours) at 50° C. and 200 mbar to generate a dried lignocellulosic material.
Drying the residue over-night (about 16 hours) at 50° C. and 200 mbar to generate a dried residue containing pretreated lignocellulosic material.
Calculating the percentage of the weight that was converted, i.e.:

Degree of liquefaction (%)=(W_{feed}−W_{residue})/W_{feed}*100%

wherein

W_{feed} is the weight (grams) of the dried lignocellulosic material used as a feed

W_{feed} is the weight (grams) of the dried residue containing the pretreated lignocellulosic material.

The indication on insoluble salts listed in table 2 can be calculated by:
Determining the pre-reaction pH of the aqueous acid solution as described above and converting this to a corresponding pre-reaction concentration of sulphuric acid (gram sulphuric acid/liter aqueous acid solution) with help of table 1 and FIG. 1;
Determining the post-reaction pH as described above and converting this to a corresponding post-reaction concentration of sulphuric acid (gram sulphuric acid/liter aqueous acid solution) with help of table 1 and FIG. 1;
Calculating the concentration of insoluble salts that can be formed upon neutralization (listed as insoluble salts in table 2), i.e.:

Insoluble salts (g/l)=[C]_{pre-reaction} (g/l)−[C]_{post-reaction} (g/l)

As illustrated in table 2, a steep decrease in insoluble salt formation occurs when a post-reaction pH of equal to or more than 3.0 is used.

In addition, the calcium content (ppmw) in milligrams (mg) per kilogram (kg) in the lignocellulosic material feed (LM feed) and in the dried residue containing the pretreated lignocellulosic material (LM residue) was determined by means of ICP-AES. The results are listed in the continuation of table 2. On the basis of calcium content in the lignocellulosic material feed and the degree of liquefaction, a theoretical 100% Calcium (Ca) content was calculated for a residue where no calcium has been leached out. Subsequently the percentage of Calcium that had leached out and the percentage of Calcium that was retained by the lignocellulosic material is determined. As illustrated in the continuation of table 2, the process according to the invention advantageously reduces the amount of calcium that is leached from the lignocellulosic material and advantageously increases the amount of calcium retained in the lignocellulosic material. As a consequence less insoluble calcium salts may be formed and less salt deposits (scale) may be formed on equipment used.

### Enzymatic Hydrolysis Step

An appropriate amount of grams, corresponding to 0.4 g cellulose, was taken from the dried pretreated lignocellulosic material obtained in pretreatment step a). This amount of dried pretreated lignocellulosic material was weighed into a 50 ml glass conical flask and to this was added 2 ml of sodium citrate solution buffered to pH=5. The total weight was then made up to 8 g with demi water. The flasks were then placed in a Stuart incubating oven fitted with a shaking table, for 30 minutes at 50° C. and shaken at 300 rpm. After 30 minutes, a fixed amount of material was removed from each flask and this material was centrifuged in a Heraeus Fresco 21 micro-centrifuge.

To determine the situation at t=0, where no enzymatic hydrolysis has taken place, 100 μl of clear liquid was pipetted from the centrifuged material and 900 μl of 10 mM H2SO4 was added to this sample. Subsequently the sample was treated in the same way as the remaining liquid and solid from the centrifuge tube.

The remaining liquid and solid from the centrifuge tube out of the Heraeus Fresco 21 micro-centrifuge were returned to the flask and then 250 μl (225 mg/g, corresponding to 45 mg protein resulting in 113 mg protein/g cellulose) of commercially obtainable Cellulase enzyme, GC-220 (Genencor International Inc.), was added to each flask and each sample. A further 2 g of demi water was added to each flask.

Both the flask and the sample for time=0 were put back into the Stuart incubating oven fitted with the shaking table, for 120 hours at 50° C. and shaken at 300 rpm. Hereafter a fixed amount of material was taken from each flask and sample and centrifuged in a Heraeus Fresco 21 micro-centrifuge. The centrifuged material was analysed in a YSI 2700 Select Biochemistry Analyzer to determine the content of glucose as listed in table 3. As illustrated in table 3 still sufficient yields of sugar can be obtained.

TABLE 2
Pretreatment of lignocellulosic material (LM)

| | | Aqueous | Solid: liquid | | Reaction | Acid conc. in the | pre- | post- | Degree | Insoluble |
|---|---|---|---|---|---|---|---|---|---|---|
| | | acid | weight | T | time | aqueous acid solution | reaction | reaction | of liquef. | salts |
| Ex | LM | solution of | ratio | (° C.) | (minutes) | gram/liter (wt %) | pH | pH | (%) | (gram/liter) |
| A | BW | H2SO4 | 1:10 | 150 | 120 | 1.09 (0.109) | 1.8 | 2.5 | 33.3 | 9.2 |
| 1 | BW | H2SO4 | 1:10 | 170 | 120 | 0.63 (0.063) | 2.0 | 3.1 | 35.8 | 5.9 |
| 2 | BW | H2SO4 | 1:10 | 150 | 120 | 0.17 (0.017) | 2.5 | 3.6 | 30.3 | 1.6 |
| 3 | WS | H2SO4 | 1:10 | 150 | 120 | 0.17 (0.017) | 2.5 | 4.5 | 24.7 | 1.7 |
| 8 | BW | H2SO4 | 1:10 | 170 | 120 | 0.17 (0.017) | 2.5 | 3.4 | 32.9 | 1.5 |
| 5 | WS | H2SO4 | 1:10 | 170 | 120 | 0.17 (0.017) | 2.5 | 3.8 | 25.6 | 1.7 |
| 6 | BW | H2SO4 | 1:10 | 170 | 120 | 0.17 (0.017) | 2.5 | 3.4 | 33.9 | 1.5 |
| B | WS | H2SO4 | 1:10 | 170 | 120 | 2.70 (0.270) | 1.5 | 2.5 | 37.8 | 25.5 |
| C | WS | H2SO4 | 1:10 | 150 | 120 | 2.70 (0.270) | 1.5 | 2.4 | 56.4 | 24.8 |
| D | BW | H2SO4 | 1:10 | 170 | 120 | 0.83 | 1.9 | 2.9 | 35.4 | 7.7 |
| E | BW | H2SO4 | 1:10 | 150 | 120 | 0.83 | 1.9 | 2.8 | 31.9 | 7.4 |
| F | WS | H2SO4 | 1:10 | 150 | 120 | 2.70 (0.270) | 1.5 | 2.4 | 19.2 | 24.7 |
| G | WS | H2SO4 | 1:10 | 150 | 120 | 2.70 (0.270) | 1.5 | 2.4 | 35.6 | 24.8 |
| 7 | WS | H2SO4 | 1:10 | 170 | 120 | 0.17 (0.017) | 2.5 | 4.0 | 32.5 | 1.7 |
| J | WS | H2SO4 | 1:10 | 80 | 60 | 4.11 (0.411) | 1.3 | 1.7 | 15.0 | 25.6 |
| K | WS | H2SO4 | 1:10 | 80 | 60 | 3.91 (0.391) | 1.3 | 1.9 | 14.8 | 30.7 |

| | | Ca- content in | Degree of | Theoretical 100% | Measured Ca- | | % Ca |
|---|---|---|---|---|---|---|---|
| | | LM feed* | liquef. | Ca- content in LM | content in LM | % Ca | recovered in |
| Ex | LM | (mg/kg) | (%) | residue* (mg/kg) | residue* (mg/kg) | leached out | residue |
| A | BW | | | — | — | — | — |
| 1 | BW | 4365 | 35.8 | 6799 | 4045 | 40.5 | 59.5 |
| 2 | BW | 4365 | 30.3 | 6263 | 4220 | 32.6 | 67.4 |
| 3 | WS | 1695 | 24.7 | 2251 | 1965 | 12.7 | 87.3 |
| 8 | BW | | | — | — | — | — |
| 5 | WS | | | — | — | — | — |
| 6 | BW | | | — | — | — | — |
| B | WS | 1695 | 37.8 | 2725 | 390 | 85.7 | 14.3 |
| C | WS | 1695 | 56.4 | 3888 | 999 | 74.3 | 25.7 |
| D | BW | | | — | — | — | — |
| E | BW | | | — | — | — | — |
| F | WS | | | — | — | — | — |
| G | WS | 1695 | 35.6 | 2632 | 1055 | 59.9 | 40.1 |
| 7 | WS | | | — | — | — | — |
| J | WS | 1695 | 15.0 | 1994 | 180 | 91.0 | 9.0 |
| K | WS | | | — | — | — | — |

wherein BW = birch wood;
WS = wheat straw;
FA = formic acid;
H2SO4 = sulphuric acid
“—” = not measured
*ppmw on a dry basis as determined via ICP-AES

TABLE 3
Enzymatic hydrolysis of pretreated lignocellulosic material

| | Enzym. Hydrol. | Enzym. Hydrol. |
|---|---|---|
| Example | glucose (g/liter) | (% of theory) |
| A | 25.7 | 46.6 |
| 1 | 34.6 | 62.8 |
| 2 | 28.0 | 50.9 |
| 3 | 29.7 | 54.0 |
| 8 | — | — |
| 5 | 47.3 | 86.1 |
| 6 | 31.3 | 56.9 |
| B | 49.3 | 89.6 |
| C | — | — |
| D | 32.8 | 59.7 |
| E | 27.9 | 50.8 |
| F | — | — |
| G | 41.0 | 74.6 |
| 7 | 40.7 | 73.9 |
| J | — | — |
| K | — | — |

“—” = not determined
% of theory = % of theoritical 100% if all cellulose would have been converted to glucose.

What is claimed is:

1. A method for processing a lignocellulosic material comprising the steps of
a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution, having an overall pH in the range from equal to or more than 3.0 to equal to or less than 4.5; and
b) contacting at least a portion of the mixture produced in step a) with a base to produce a neutralized mixture containing neutralized pretreated lignocellulosic material and one or more insoluble salts.

2. A process for the production of at least one alkanol comprising the steps of:
a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution, having an overall pH in the range from equal to or more than 3.0 to equal to or less than 4.5;
b) optionally contacting at least a portion of the mixture produced in step a) with a base to produce a neutralized mixture containing neutralized pretreated lignocellulosic material and one or more insoluble salts;
c) hydrolyzing at least a portion of the pretreated lignocellulosic material produced in step a) and/or at least a portion of the neutralized pretreated lignocellulosic material produced in step b) to produce a hydrolysis product; and
d) fermenting at least a portion of the hydrolysis product produced in step c) to produce a fermentation broth comprising at least one alkanol.

3. The process of claim 2 further comprising step e) recovering the alkanol from the fermentation broth produced in step d).

4. The method of claim 1 wherein the lignocellulosic material is a lignocellulosic material containing equal to or more than 100 ppmw of calcium, based on the total weight of lignocellulosic material on a dry basis.

5. The process of claim 2 wherein the lignocellulosic material is a lignocellulosic material containing equal to or more than 100 ppmw of calcium, based on the total weight of lignocellulosic material on a dry basis.

6. The method of claim 1 wherein step a) is carried out in the essential absence of an external base.

7. The method of claim 1 wherein the aqueous acid solution is an aqueous acid solution of one or more inorganic acid(s), which aqueous acid solution contains essentially no organic acid(s).

8. The method of claim 1 wherein the aqueous acid solution is an aqueous acid solution of sulphuric acid.

9. The method of claim 1 wherein the aqueous acid solution is an aqueous acid solution that comprises in the range from equal to or more than 0.00001 wt % to equal to or less than 0.08 wt % sulphuric acid, based on the total weight of the aqueous acid solution.

10. The method of claim 1 wherein the pretreated lignocellulosic material produced in step a) contains a total amount of calcium equal to or more than 70 wt %, of the total amount of calcium in the lignocellulosic material used as a feed to step a).

11. The process of claim 2 wherein the aqueous acid solution is an aqueous acid solution of sulphuric acid.

12. The method of claim 1 further comprising a desalting step.

13. The process according to method of claim 12 wherein the desalting step comprises electrodialysis of a product containing one or more insoluble salts to produce a concentrated salt solution; and removing and/or recovering insoluble salts from the concentrated salt solution by means of crystallization.

14. The method of claim 12 wherein the desalting step comprises anaerobic fermentation of a product containing the one or more insoluble salts to produce a desalting residue containing the insoluble salts; and recovering the insoluble salts from the residue.

15. The method of claim 12 wherein the desalting step comprises contacting of a product containing the one or more insoluble salts with one or more ion-exchange resins to produce a concentrated salt solution; and removing and/or retrieving the insoluble salts from the concentrated salt solution by means of crystallization.

16. The method of claim 12 wherein the desalting step comprises:
(I) removing water from the fermentation broth produced in step d) and/or one or more distillate fraction(s) and/or one or more residue fraction(s) obtained in optional step e) by means of evaporation to produce a concentrated product;
(II) burning the concentrated product produced in step (I) to produce ashes; and
(III) removing and/or retrieving alkali metal salts and/or alkali metal earth salts from the ashes.

17. A method for processing a lignocellulosic material comprising the steps of:
a) contacting the lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution;
b) leaching basic compounds from the lignocellulosic material to adjust the overall pH of the mixture to a pH in the range from equal to or more than 3.0 to equal to or less than 4.5; and
c) contacting at least a portion of the mixture produced in step a) with a base to produce a neutralized mixture containing neutralized pretreated lignocellulosic material and one or more insoluble salts.

18. The method of claim 17 wherein the aqueous acid solution is an aqueous acid solution of sulphuric acid.

19. The method of claim 17 wherein the lignocellulosic material is a lignocellulosic material containing equal to or more than 100 ppmw of calcium, based on the total weight of lignocellulosic material on a dry basis.

20. A process for processing a lignocellulosic material comprising the steps of
a) contacting a lignocellulosic material at a temperature in the range from equal to or more than 120° C. to equal to or less than 210° C. with an aqueous acid solution containing one or more inorganic acids and having a pH in the range from equal to or more than 1.8 to equal to or less than 4.0 to produce a mixture containing pretreated lignocellulosic material and aqueous acid solution, having an overall pH in the range from equal to or more than 3.0 to equal to or less than 4.5; and
b) neutralizing the mixture produced in step a) to produce a neutralized mixture having a higher pH than the mixture produced in step a).
